(19) United States
(12) Patent Application Publication
(10) Pub. No.: US 2021/0361620 A1
(43) Pub. Date: Nov. 25, 2021

(54) SLEEP ENHANCEMENT WITH A PROTEIN-BOUND TRYPTOPHAN AND MELATONIN MIXTURE

(71) Applicant: Biosential, Inc., Toronto (CA)

(72) Inventor: Craig J. HUDSON, Tiribti (CA)

(21) Appl. No.: 17/272,188

(22) PCT Filed: Aug. 29, 2019
(86) PCT No.: PCT/IB2019/000951

§ 371 Date: Feb. 26, 2021

Related U.S. Application Data
(60) Provisional application No. 62/725,384, filed on Aug. 31, 2018.

(51) Int. Cl. A61K 31/405; A61K 31/4045; A61P 25/20; A61K 47/26; A61K 47/46; A23L 33/105; A23L 33/175; A23L 33/125; A23L 33/15; A23L 2/52

(52) U.S. Cl. CPC A61K 31/405; A61K 31/4045; A61P 25/20; A61K 47/26; A61K 47/46; A23V 2002/00; A23L 33/175; A23L 33/125; A23L 33/15; A23L 2/52; A23L 33/105

(57) ABSTRACT

A composition comprising, preferably, at least partially defatted meal from a plant source containing protein-bound tryptophan that, preferably, has a higher tryptophan source than said plant source, a melatonin component, and a pharmaceutically-acceptable diluent or carrier therefor for enhancing sleep quality and duration in a mammal. In some embodiments, the composition includes a carbohydrate. The invention provides a method of enhancing sleep quality in a mammal having a composition described above.

### FIELD OF THE INVENTION

This invention describes a composition that contains natural sources of protein-bound tryptophan from plants, natural plant sources of melatonin, processes for creating said compositions, physical formulations of said compositions, and use of said compositions for enhancing sleep quality and duration in mammals.

### BACKGROUND OF THE INVENTION

Tryptophan is an essential amino acid found in many naturally-occurring plant proteins. After ingestion and absorption, tryptophan travels through the circulatory system as approximately 80% bound to serum albumin with the remaining 20% as free tryptophan. Under certain conditions tryptophan is transported into the brain and then metabolized through several biochemical pathways, one of which produces serotonin—a major neurotransmitter involved in the regulation of mood, anxiety and sleep (Pardridge, 1979). In darkness serotonin is further metabolized to melatonin—a neurohormone that regulates the sleep-wake cycle.

Melatonin is primarily synthesized in and secreted by the pineal gland—a small cone-like structure in the epithalamus of the brain (Wurtman, Axelrod & Fischer, 1964). During the night, the suprachiasmatic nucleus (SCN) of the hypothalamus stimulates the pineal gland to produce and secrete melatonin, which then acts on MT1 and MT2 receptors throughout the body to promote sleep (Dubocovich, 1988). Melatonin synthesis is regulated by feedback inhibition; at its peak concentration, melatonin suppresses SCN activity to inhibit its further synthesis, thereby regulating its own levels in the brain (Bedrosian et al., 2013). Melatonin synthesis and release therefore follow a circadian pattern, with a peak amplitude plasma concentration maintained at 25-120 pg/mL throughout the night (Waldhauser & Dietzel, 1985; Lewy & Newsome, 1983).

The hypnotic effects of both tryptophan and melatonin supplementation are well-studied. The ingestion of a sufficient quantity of tryptophan per se consistently reduces sleep latency, i.e. the time taken to fall asleep from “lights out”, and improves sleep quality through enhanced sleep architecture (Boman, 1988). The hypnotic effect of tryptophan follows a dose-response curve, on which a minimum of 250 mg can improve sleep in subjects with mild insomnia or longer-than-average sleep latency (Hartmann and Spinweber, 1976; Hartmann, 1982); however, doses of 1000 mg produce more consistent improvements on sleep quality (Schneider-Helmut and Spinweber, 1986). Higher doses of tryptophan (i.e. 2,000-12,000 mg) produce little extra benefit, and doses above 12,000 mg have shown to disrupt sleep architecture (Griffiths et al., 1972). Likewise, the ingestion of a sufficient quantity of melatonin has been shown to reduce sleep latency, increase sleep duration and efficiency, and regulate the sleep-wake cycle (Dawson & Encel, 1993). The sleep-promoting effects of melatonin also exhibit a dose-response relationship, where modest increases above 1 to 5 mg produce minimal benefit. (Sack et al., 1997; Dawson & Encel, 1993). Melatonin is currently sold as an over-the-counter dietary supplement in North America.

Despite their individual hypnotic effects, the combination of tryptophan and melatonin is not expected to have a synergistic benefit on sleep due to melatonin feedback inhibition. Exogenous melatonin should theoretically suppress further endogenous melatonin synthesis to maintain an optimal plasma concentration. We have found, however, that the combined ingestion of protein-bound tryptophan and melatonin produced an unexpected superior benefit on subjective sleep quality to protein-bound tryptophan alone. The addition of 1 mg melatonin per 10,000 mg composition described in aforesaid WO 01/89319 has engendered improved sleep benefits compared to the previous melatonin-free mixture. Based on self-report data from 14 participants, 100% experienced reduced sleep latency and/or increased sleep duration, and 29% of participants reported more regular sleep-wake cycles. Yet further, there are no reports to date on the synergistic effect of tryptophan-bound protein with melatonin supplementation on sleep quality enhancement.

### SUMMARY OF THE INVENTION

We have surprisingly discovered that the addition of plant-derived melatonin to the protein-bound tryptophan composition defined in WO 01/89319 produces enhanced sleep quality and duration in a mammal.

We have surprisingly discovered that human subjects who ingested a high protein food composition rich in both protein-bound tryptophan and plant-derived melatonin reported an increased improvement in sleep quality and duration, and decreased sleep latency, compared to protein-bound tryptophan alone. These results were obtained from self-report surveys for subjects comparing the sleep benefits before and after the addition of 1 mg of melatonin per 10,000 mg of the composition defined in WO 01/89319.

Thus, it is an object of this invention to provide plant sources of tryptophan and melatonin for combined use to enhance sleep quality and duration in mammals.

The invention provides a plant source of protein-bound tryptophan, a plant source of melatonin, and a physiologically acceptable diluent or carrier therefore for enhancing sleep quality and duration in mammals. Most preferably, the meal is at least partially defatted.

The addition of plant-derived melatonin to the naturally-derived tryptophan-rich composition described in aforesaid WO 01/89319 has been found to enhance sleep quality and duration in mammals. Compositions of use in the invention comprise a plant source naturally containing protein-bound tryptophan—preferably squash seeds such as butternut squash seeds, peppercorn squash seeds and pumpkin seeds. Preferably, the plant source of protein-bound tryptophan is at least partially defatted. Compositions of use in the invention also comprises a plant source naturally containing melatonin—preferably pigmented rice grains. The composition also preferably contains a carbohydrate source, such as glucose, in an amount sufficient to facilitate the transport of tryptophan across the blood-brain barrier (BBB) and to circumvent the large neutral amino acid (LNAA) competition for BBB transport sites into the central nervous system (CNS). The composition can also optionally include physiologically acceptable vehicle(s), flavorings, colours and other nutrients such as vitamins B3 and/or vitamin B6.

The preferred composition of the present invention includes at least partially defatted squash seeds, particularly butternut squash, pumpkin and peppercorn squash seeds, pigmented rice grains, glucose and vitamins B3 and B6.

The invention further pertains to dietary supplements in the form of a drink mixture comprising the composition of the invention.

According to WO 01/89319, the use of second derivative spectroscopy has found that certain plant sources and specifically plant seeds contain relatively high levels of protein-bound tryptophan, and that these sources can provide tryptophan in vivo.

The use of analytical pressurized liquid extraction with high-performance liquid chromatography (HPLC) has found high levels—above 100 μg/kg—of melatonin in various types of pigmented rice grains, and particularly in polished, whole grain, aromatic, black, black glutinous, red and parboiled rice grains (Setyaningsih et al., 2015). Red rice grains contain high levels of melatonin and can be used herein as the plant source. Preferably, the starting rice grains contain at least 1 mg of melatonin. Melatonin concentration can be determined using known analytic methods, including, for example, HPLC and microwave-assisted extraction (MAE) (Setyaningsih, Palma & Barroso, 2012).

The plant source of melatonin can be, for example, but not limited to, pigmented rice, wheat, barley, oats, bread crumbs, grapes, cherries, strawberries, tomatoes, peppers, mushrooms, germinated white mustard seeds, germinated black mustard seeds and nuts. Preferably, the plant source is red rice grain as it is believed to contain the highest concentration of melatonin relative to other types of pigmented rice grains.

(The plant source can also be medicinal herbs including, but not limited to, Huang-qin and St. John's Wort flowers and leaves).

The composition of use in the practice of the invention preferably includes a high-glycemic index carbohydrate. The carbohydrate is preferably in the form of glucose, but may also be sucrose and other sugars that metabolize into glucose. It has been found that glucose facilitates the transport of tryptophan across the blood-brain barrier (BBB) for further metabolism into serotonin and melatonin in the central nervous system (CNS). The BBB consists of tight junctions between cerebral endothelial cells that regulate the diffusion of substances into the CNS. A series of transport mechanisms within the BBB selectively facilitate the movement of particular molecules into the CNS. The large neutral amino acid transporter (LAT) is one such mechanism that transports large neutral amino acids (LNAAs), including tryptophan, across the BBB. LNAAs compete for access to LAT, which prevents tryptophan transport to the brain. High-glycemic index carbohydrates can facilitate the uptake of tryptophan across the BBB relative to other LNAAs. The carbohydrate triggers the release of insulin, which shunts circulating LNAAs into skeletal muscle. Tryptophan, however, is not shunted, and remains in the bloodstream to more easily access LAT for transport across the BBB.

The composition of the invention is to be orally administered daily. The composition is formulated for the intended use of a single daily administration prior to bedtime in the absence of daylight.

It is noted that tryptophan supplementation has been shown to have potential side effects including drowsiness and minimal weight loss.

Serious side effects of short-term melatonin supplementation have not been reported in humans to date. Although uncommon, 1 mg to 5 mg of melatonin has been shown to produce drowsiness, headache, dizziness and nausea (Andersen et al., 2016). Melatonin supplementation during the day may result in sleepiness and hypothermia, which may be avoided with nighttime use (Cagnacci, Elliott & Yen, 1992). At nanomolar concentrations, melatonin has been shown to produce a vasoconstricting effect, whereas micromolar or millimolar concentrations of melatonin have been shown to produce vasodilation (Guardiola-Lemaitre, 1997); thus, the use of the composition in patients with cardiovascular disease should be at the discretion of a physician. The combination of melatonin with anti-inflammatory treatments may also increase the risk of developing gastric ulcerations (Guardiola-Lemaitre, 1997). Melatonin supplementation has also been shown to alter glucose metabolism by exerting an antihyperglycemic effect in rats; thus, individuals with Type I or Type II diabetes for which they receive chronic treatment of insulin or other means should be monitored by a physician for alterations in glucose metabolism (Guardiola-Lemaitre, 1997). The side effects of long-term melatonin supplementation requires further investigation.

### DETAILED DESCRIPTION OF PREFERRED EMBODIMENTS

### EXAMPLES

The procedures for obtaining compositions of use in the practise of the present invention are as described in aforesaid WO 01/89319.

A drink mixture of 10 grams for use according to the invention was made according to the general procedures described in aforesaid WO 01/89319 with the addition of plant-derived melatonin, having the composition:
5 gm of defatted squash seed
4.39 gm glucose (“glucose 43”)
0.49 gm Chocolate flavor
0.07 gm guar gum
0.04 gm rice starch
0.001 gm plant source melatonin

### Patient Reports

We have found, however, that the combined ingestion of protein-bound tryptophan and melatonin produced an unexpected superior benefit on subjective sleep quality to protein-bound tryptophan alone. The addition of 1 mg melatonin to 10,000 mg of a protein-bound tryptophan composition described in aforesaid WO 01/89319 has produced surprising improvements in sleep quality and duration. Based on self-report data from 14 consumers, 100% experienced reduced sleep latency and/or increased sleep duration, and 29% reported more regular sleep-wake cycles with the melatonin-supplemented composition compared to the melatonin-free composition. Several self-reports also mentioned more intense dreams, increased feelings of restfulness and deeper sleeps.

Prior to treatment with 1 mg melatonin to 10,000 mg of a protein-bound tryptophan composition, these individuals all reported long-standing insomnia and a desire to treat their insomnia with methods other than sleep medications

### EXAMPLES

One survey participant, a middle aged man from Bavaria, Germany reported issues with falling and staying asleep when consuming the melatonin-free protein-bound tryptophan composition once per day. The participant then consumed 10,000 mg of the composition with 0.01% added melatonin once before bed. After 60 days of this consumption, the participant reported decreased sleep latency and increased sleep duration. The participant also reported that other sleep-enhancing supplements and hypnotic medications were no longer necessary to remediate his or her insomnia-related issues.

Another participant, a middle aged woman from Germany reported a reduced quality of life due to drowsiness and long periods of wakefulness when regularly consuming the melatonin-free tryptophan composition. After 60 days of consuming 10 g of the 0.01% melatonin composition once before bed, the participant reported quick improvements in both sleep quality and ease of waking up in the morning.

Another German woman participant had struggled to fall asleep while regularly taking the melatonin-free tryptophan composition. The participant then consumed 10 g of the 0.01% melatonin composition once before bedtime for 60 days. The participant then reported reduced sleep latency, improved sleep quality, and longer and more intense sleep phases.

### REFERENCES

Bedrosian, T. A., Herring, K. L., Walton, J. C., Fonken, L. K., Weil, Z. M., & Nelson, R. J. (2013). Evidence for feedback control of pineal melatonin secretion. Neuroscience letters, 542, 123-125.
Boman, B. (1988). L-tryptophan: a rational anti-depressant and a natural hypnotic?. Australian and New Zealand journal of psychiatry, 22(1), 83-97.
Cagnacci A, Elliott J A, Yen S S. Melatonin: a major regulator of the circadian rhythm of core temperature in humans. J Clin Endocrinol Metab 1992; 75: 447-52.
Dawson, D., & Encel, N. (1993). Melatonin and sleep in humans. Journal of pineal research, 15(1), 1-12.
Dubocovich, M. L. (1988). Pharmacology and function of melatonin receptors. The FASEB Journal, 2(12), 2765-2773.
Klein, D. C., Coon, S. L., Roseboom, P. H., Weller, J. L., Bernard, M., Gastel, J. A., . . . & Falcon, J. (1997). The melatonin rhythm-generating enzyme: molecular regulation of serotonin N-acetyltransferase in the pineal gland. Recent progress in hormone research, 52, 307-57.
Lewy, A. J., & Newsome, D. A. (1983). Different types of melatonin circadian secretory rhythms in some blind subjects. The Journal of Clinical Endocrinology & Metabolism, 56(6), 1103-1107.
McIntyre, I. M., Norman, T. R., Burrows, G. D., & Armstrong, S. M. (1989). Human melatonin suppression by light is intensity dependent. Journal of pineal research, 6(2), 149-156.
Sack, R. L., Hughes, R. J., Edgar, D. M., & Lewy, A. J. (1997). Sleep-promoting effects of melatonin: at what dose, in whom, under what conditions, and by what mechanisms? Sleep, 20(10), 908-915.
Setyaningsih, W., Palma, M., & Barroso, C. G. (2012). A new microwave-assisted extraction method for melatonin determination in rice grains. Journal of cereal science, 56(2), 340-346.
Waldhauser, F., & Dietzel, M. (1985). Daily and annual rhythms in human melatonin secretion: role in puberty control. Annals of the New York Academy of Sciences, 453(1), 205-214.
Wurtman, R. J., Axelrod, J., & Fischer, J. E. (1964). Melatonin synthesis in the pineal gland: effect of light mediated by the sympathetic nervous system. Science, 143(3612), 1328-1329.

1. A composition comprising a meal from a plant source containing protein-bound tryptophan, an herb containing melatonin and a physiologically-acceptable diluent or carrier therefor for enhancing sleep in mammals.

2. A composition as defined in claim 1 wherein said meal is at least partially defatted and has a higher tryptophan source than said plant source.

3. A composition as defined in claim 1 comprising a plant-derived melatonin.

4. A composition as defined in claim 1 further comprising a carbohydrate source having a high-glycemic index.

5. A composition as defined in claim 1 wherein the plant source is a seed.

6. A composition as defined in claim 5 wherein the seed is selected from the group consisting of butternut squash seed, peppercorn squash seed, pumpkin seed, lentil seed, sunflower seed, flax seed, watermelon seed, sisymbrium seed, cotton seed, sesame seed, canola seed, evening primrose seed, safflower seed, alfalfa seed, barley, soy bean and combinations thereof.

7. A composition as defined in claim 1 wherein the plant source is selected from the group consisting of seaweed, kept and alfalfa seeds.

8. A composition as defined in claim 3 wherein the plant source is a cereal.

9. A composition as defined in claim 8 wherein the cereal is selected from the group consisting of pigmented rice grains including polished, whole grain, aromatic, black, black glutinous, red and parboiled rice grains.

10. A composition as defined in claim 4 wherein the carbohydrate source is selected from the group consisting of glucose, maltose, sucrose and combinations thereof.

11. A composition as defined in claim 1 further comprising a vitamin selected from the group consisting of vitamin B3, B6 and combinations thereof in an amount sufficient to enhance uptake of the tryptophan across the blood-brain barrier.

12. A composition as defined in claim 1 in the form of a tablet, powder, suspension, liquid, capsule or gel.

13. A composition as defined in claim 1 in the form of a dietary supplement.

14. A dietary supplement as defined in claim 13 wherein the supplement is formulated in a plurality of oral dosages for ingestion on a daily basis.

15. The dietary supplement as defined in claim 14 wherein the supplement is provided as a powder drink mixture.

16. A composition as defined in claim 6 comprising at least partially defatted butternut squash seed meal having protein-bound tryptophan in an amount of 25 mg to 1000 mg of tryptophan, from 25 mg to 200 mg of glucose, and a physiologically acceptable diluent or carrier therefor.

17. A composition as defined in claim 6 comprising at least partially defatted butternut squash seed meal providing from 25 mg to 50 mg of tryptophan, from 3 μg to 12 mg of melatonin, from 75 mg to 100 mg of glucose, and a physiologically acceptable diluent or carrier therefor.

18. A composition as defined in claim 11 further comprising from 5 mg to 50 mg of vitamin B3, from 0.5 mg to 50 mg of vitamin B6, and combinations thereof.

19. (canceled)

20. A method of enhancing sleep in a mammal comprising administering an effective amount of a composition as defined in claim 11 said mammal.

21. Use of a composition as defined in claim 11 for enhancing sleep in a mammal.

22. (canceled)

23. (canceled)
